(19) United States
(12) Patent Application Publication
(10) Pub. No.: US 2019/0209130 A1
(43) Pub. Date: Jul. 11, 2019

(54) Real-Time Sagittal Plane Navigation in Ultrasound Imaging

(71) Applicant: BK Medical Holding Company, Inc., Peabody, MA (US)

(72) Inventors: David Lieblich, Worcester, MA (US); Spiros Mantzavinos, Lynn, MA (US)

(73) Assignee: BK Medical Holding Company, Inc., Peabody, MA (US)

(21) Appl. No.: 16/302,211

(22) PCT Filed: May 16, 2016
(86) PCT No.: PCT/US2016/032655

§ 371 Date: Nov. 16, 2018

(51) Int. Cl. A61B 8/00; A61B 8/08; A61B 8/12; G06K 9/62

(52) U.S. Cl. CPC A61B 8/4245; A61B 8/483; A61B 8/08; A61B 8/12; A61B 8/523; G06K 2209/05; A61B 8/466; A61B 8/463; A61B 8/4209; G06K 9/6201; G06K 2009/6213; A61B 8/4488

(57) ABSTRACT

A method includes obtaining a 3-D volume of anatomy including at least the structure of interest. The method further includes acquiring, with an array of an ultrasound probe, a real-time 2-D ultrasound image of the structure of interest in a cavity parallel to a longitudinal axis of the ultrasound probe. The method further includes calculating a metric from a 2-D plane extracted from the 3D volume and the real-time 2-D ultrasound sagittal image. The metric identifies a plane, from sagittal planes of the 3D volume, and a position within the plane, that best fits the real-time 2-D ultrasound sagittal image. The method further includes identifying a current location of the ultrasound probe with respect to the anatomy based on the identified position.

### TECHNICAL FIELD

The following generally relates to image based navigation in ultrasound and more particularly to employing real-time sagittal planes for image based navigation in ultrasound imaging.

### BACKGROUND

An ultrasound imaging system has included a probe with transducer array that transmits an ultrasound beam into an examination field of view. As the beam traverses structure (e.g., in an object or subject) in the field of view, sub-portions of the beam are attenuated, scattered, and/or reflected off the structure, with some of the reflections (echoes) traversing back towards the transducer array. The transducer array receives the echoes, which are processed to generate one or more images of the structure.

The resulting ultrasound images have been used to guide procedures in real-time, i.e., using presently generated images from presently acquired echoes. This has included registering a real-time 2-D ultrasound image to a corresponding plane in previously generated 3-D navigation anatomical image data and displaying the 3-D navigation anatomical image data with the real-time 2-D ultrasound image superimposed or overlaid over the corresponding plane. The displayed image data indicates a location and orientation of the transducer array with respect to the anatomy in the 3-D navigation anatomical image data.

The probe has been navigated for the real-time acquisition using a stabilizing arm. With a stabilizing arm, accurate positioning has required a manual gear mechanism to translate and rotate about the axis of the arm, and possibly other axes as well. This approach is subject to human error. To mitigate such error, some arms come with encoders to automate the recording of position. However, encoders further increase the cost of the arm and require additional time and expertise in setup and use. Another approach is freehand navigation (i.e., no stabilizing arm). However, freehand navigation based on an external navigation system (for example, optical, magnetic, and/or electromagnetic) adds components, which increases overall complexity and cost of the system.

### SUMMARY

Aspects of the application address the above matters, and others.

According to one aspect, a method includes obtaining a 3-D volume of anatomy including at least the structure of interest. The method further includes acquiring, with an array of an ultrasound probe, a real-time 2-D ultrasound image of the structure of interest in a cavity parallel to a longitudinal axis of the ultrasound probe. The method further includes calculating a metric from a 2-D plane extracted from the 3D volume and the real-time 2-D ultrasound sagittal image. The metric identifies a plane, from sagittal planes of the 3D volume, and a position within the plane, that best fits the real-time 2-D ultrasound sagittal image. The method further includes identifying a current location of the ultrasound probe with respect to the anatomy based on the identified position.

In another aspect, an apparatus includes a sagittal or end-fire transducer array of an ultrasound probe, wherein the sagittal or end-fire transducer array is configured to transmit and receive echoes. The apparatus further includes a beamformer configured to process the echoes and generate a real-time 2-D sagittal or endfire ultrasound image. The apparatus further includes a navigation processor configured to calculate a metric, from a 2-D plane extracted from a 3D volume and the real-time 2-D ultrasound sagittal or endfire image, to identify a plane, from sagittal or endfire planes of the 3D volume, and a position within the plane, that best fits the real-time 2-D ultrasound sagittal or endfire image.

In another aspect, a non-transitory computer readable medium is encoded with computer executable instructions, which, when executed by a computer processor, causes the processor to: calculate a metric from a 2-D plane extracted from the 3D volume and a real-time 2-D ultrasound sagittal image, wherein the metric identifies a plane, from sagittal planes of the 3D volume, and a position within the plane, that best fits the real-time 2-D ultrasound sagittal image, and identify a current location of the ultrasound probe based on the identified position.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example ultrasound imaging system with a navigation processor sagittal or endfire plane matching component;

FIG. 2 schematically illustrates an example of the navigation processor;

FIG. 3 illustrates an example method using a probe support to position and move the probe for a procedure; and

FIG. 4 illustrates an example method with freehand movement of the probe to position and move the probe for a procedure.

### DETAILED DESCRIPTION

The following generally describes an approach for stabilizing arm and/or freehand based navigation in which real-time 2-D sagittal plane ultrasound images are acquired by rotating and translating the probe and matched to a sagittal plane in a reference 3-D volume to determine an offset(s) of the current plane from a reference location in the 3-D volume. As utilized herein, a real-time 2-D ultrasound image refers to a currently or presently generated image, generated from echoes currently or presently acquired.

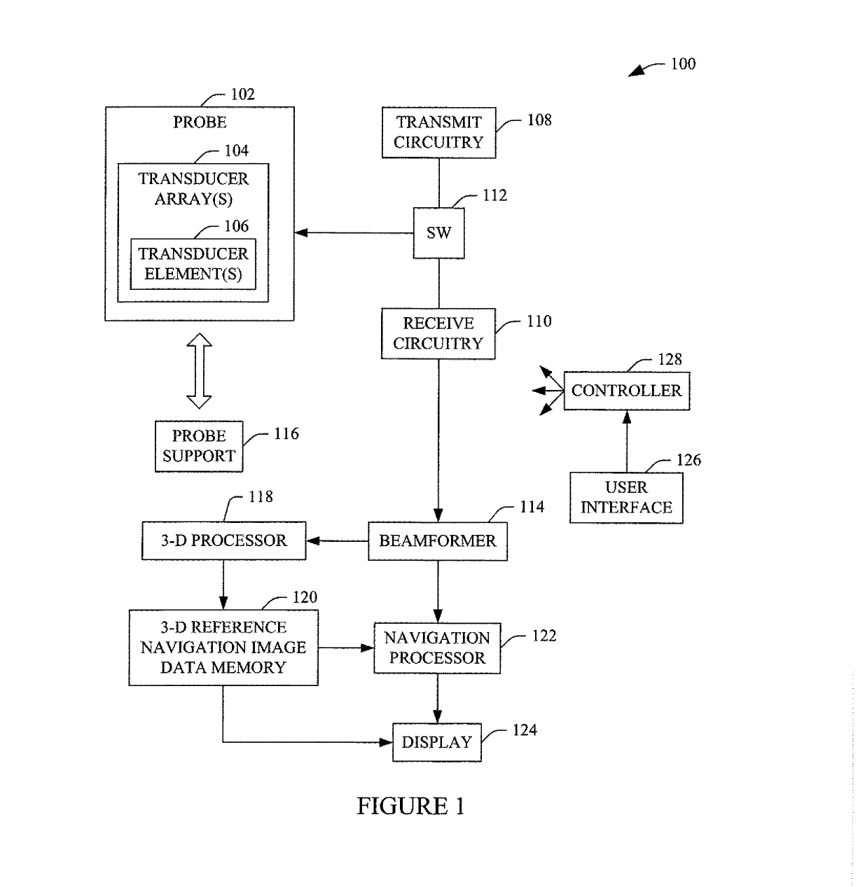

Initially referring to FIG. 1, an ultrasound imaging system 100 includes a probe 102 housing a transducer array 104 having at least one transducer element 106. The at least one transducer element 106 is configured to convert electrical signals to an ultrasound pressured field and vice versa respectively to transmit ultrasound signals into a field of view and receive echo signals, generated in response to interaction with structure in the field of view, from the field of view. The illustrated transducer array 104 can include one or more arrays, including linear, curved (e.g., concave, convex, etc.), circular, etc. arrays, which are fully populated or sparse, etc. Examples of suitable probes 102 include a sagittal probe, an end-fire probe, a biplane probe with both a sagittal and an axial array, and/or other probes configured to acquire data in a plane parallel to the long axis of the ultrasound probe.

Transmit circuitry 108 generates a set of pulses (or a pulsed signal) that are conveyed, via hardwire (e.g., through a cable) and/or wirelessly, to the transducer array 104. The set of pulses excites a set (i.e., a sub-set or all) of the at least one transducer element 106 to transmit ultrasound signals. Receive circuitry 110 receives a set of echoes (or echo signals) generated in response to a transmitted ultrasound signal interacting with structure in the field of view. A switch (SW) 112 controls whether the transmit circuitry 108 or the receive circuitry 110 is in electrical communication with the at least one transducer element 106 to transmit ultrasound signals or receive echoes.

A beamformer 114 processes the received echoes by applying time delays to echoes, weighting echoes, summing delayed and weighted echoes, and/or otherwise beamforming received echoes, creating beamformed data. In B-mode imaging, the beamformer 114 produces a sequence of focused, coherent echo samples along focused scanlines of a scanplane. The scanplanes correspond to the plane(s) of the transducer array(s) 104. The beamformer 114 may also process the scanlines to lower speckle and/or improve specular reflector delineation via spatial compounding, and/or perform other processing such as FIR filtering, IIR filtering, edge enhancement, etc.

A probe support 116 is configured to support the probe 102, including translate and/or rotate the probe 102. This includes translating and/or rotating the probe 102 during a procedure to position the probe 102 with respect to structure of interest to acquire data for a set of planes, such as one or more sagittal planes. This includes positioning the probe 102 at a location where the structure(s) of interest is imaged within the 2D plane and planes spanning an angular range that completely encloses the volume(s) of the structure(s) of interest, followed by a real-time scan of the region with standard 2D ultrasound acquisition. This 3D sweep and the subsequent real-time scan may be accomplished freehand or with a probe support.

A 3-D processor 118 generates 3-D navigation image data, and 3-D navigation image data memory 120 stores the 3-D navigation image data. In this example, the 3-D processor 118 processes 2-D images from the beamformer 114 to generate the 3-D reference navigation image data. The 2-D images can be acquired using a freehand and/or other approach and include a set of images sampling the structure(s) of interest and spanning the full extent of its volume(s). In one instance, sagittal images spanning an angular range are correctly distributed within their collected angular range and combined to produce the 3-D navigation image data based upon detected angles of axial rotation of the probe 102, e.g., determined from axial images generated from data acquired from an axial array of a biplane probe, or a displacement signal from a motion sensor of the probe 102.

In general, the 3-D navigation image data is acquired sufficiently slow to acquire a dense angular array of slices in the sagittal or endfire plane. Generally, the depth of penetration can be set sufficiently large to encompass the entire structure of interest, throughout the extent of its volume, while maintaining sufficient resolution, within each sagittal or endfire plane. When this is not possible, one or more acquisitions at one or more different locations are performed to cover an entire extent of a structure of interest. The data from the different acquisitions is registered, e.g., using overlap regions, and may be interpolated to produce a dense Cartesian volume, or used in its original form. A non-limiting example of generating a 3-D volume from 2-D images acquired using a freehand probe rotation or translation is described in patent application serial number PCT/US2016/32639, filed May 16, 2016, entitled “3-D US VOLUME FROM 2-D IMAGES FROM FREEHAND ROTATION OR TRANSLATION OF ULTRASOUND PROBE,” the entirety of which is incorporated herein by reference.

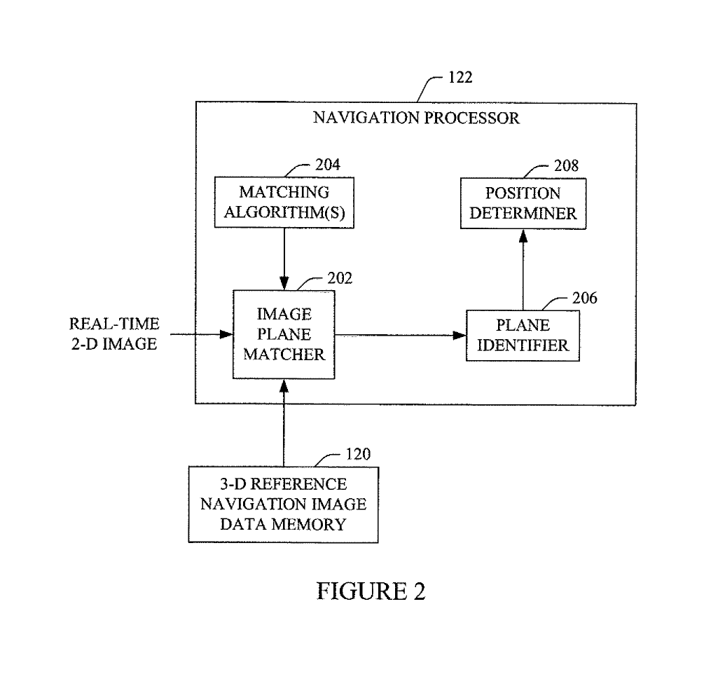

A navigation processor 122 maps a real-time 2-D sagittal ultrasound image generated by the imaging system 100 to a corresponding image plane in the 3-D navigation image data. The real-time 2-D sagittal ultrasound image can be generated with a sagittal or end-fire probe or a sagittal array of a biplane probe. Various approaches can be used for the matching such as a similarity algorithm. FIG. 2 shows an example in which the navigation processor 122 includes a matcher 202 and at least one matching algorithm(s) 204. In this example, the at least one matching algorithm(s) 204 includes a similarity algorithm such as at least a normalized cross-correlation algorithm and/or other algorithm. The matcher 202 employs the algorithm to match the real-time 2-D sagittal ultrasound image with sagittal planes of the 3-D navigation image data. A plane identifier 206 identifies a plane of the 3-D navigation image data that represents a best fit with the real-time 2-D sagittal ultrasound image, as for example, the peak value of a matching metric and/or values of the matching metric that surpass a threshold value. In the event of ambiguity amongst a number of planes, the plane that provides the best continuity to at least one prior position is selected. A position determiner 208 determines a radial angle based on the plane of best fit and a translational offset within the plane of best fit, when a probe support is used. When a freehand scan is performed, the position determiner 208 determines the normal to the plane of best fit and a reference point in the plane corresponding to a known reference point in the 2D sagittal ultrasound image, for example the upper left corner, which positions the 2D sagittal ultrasound image within the 3D volume.

The number of planes of the 3-D navigation image data matched to the real-time 2-D sagittal ultrasound image can be reduced by, e.g., using the angle, or approximation thereof, derived from an axial image, e.g., where the probe 102 is a biplane probe, and/or other estimate of probe orientation such as a limited angular range covering the prostate in the case of a probe support or an approximate position and orientation in the case of a freehand probe, e.g., where no axial image exists. When an axial plane is available, e.g., where the probe 102 is a biplane probe, the axial plane can also be used to obtain an independent check of the location by measuring its similarity to the corresponding axial plane, if one exists, or can be interpolated from the 3D data, at the offset determined by the sagittal plane. In a variation, anatomical structure is segmented in the 3-D navigation image data and corresponding anatomical structure is segmented in the real-time 2-D sagittal ultrasound image by the navigation processor 122 and/or other component, and common segmented anatomical structure segmented in both data sets is additionally or alternatively matched to identify the plane of best fit.

Returning to FIG. 1, in one instance the real-time 2-D ultrasound image is superimposed over the 3-D reference navigation image at the matched image plane based on the position and visually presented via a display 124. In a variation, graphical indicia (e.g., an arrow, a schematic of the probe, etc.) is overlaid over the 3-D reference navigation image data at the matched image plane based on the position. The resulting combination identifies the location and/or orientation of the ultrasound transducer 104 relative to the current location of the probe 102 with respect to the anatomy in the 3-D navigation image data, which allows a clinician to use the real-time 2-D ultrasound image and the 3-D navigation image data to navigate to tissue of interest in the scanned anatomy.

A user interface (UI) 126 includes an input device(s) (e.g., a physical button, a touch screen, etc.) and/or an output device(s) (e.g., a touch screen, a display, etc.), which allow for interaction between a user and the ultrasound imaging system 100. A controller 128 controls one or more of the components 102-126 of the ultrasound imaging system 100. Such control includes controlling one or more of the components to perform the functions described herein and/or other functions.

In the illustrated example, at least one of the components of the system 100 (e.g., the navigation processor 122) can be implemented via one or more computer processors (e.g., a microprocessor, a control processing unit, a controller, etc.) executing one or more computer readable instructions encoded or embodied on computer readable storage medium (which excludes transitory medium), such as physical computer memory, which causes the one or more computer processors to carry out the various acts and/or other functions and/or acts. Additionally or alternatively, the one or more computer processors can execute instructions carried by transitory medium such as a signal or carrier wave.

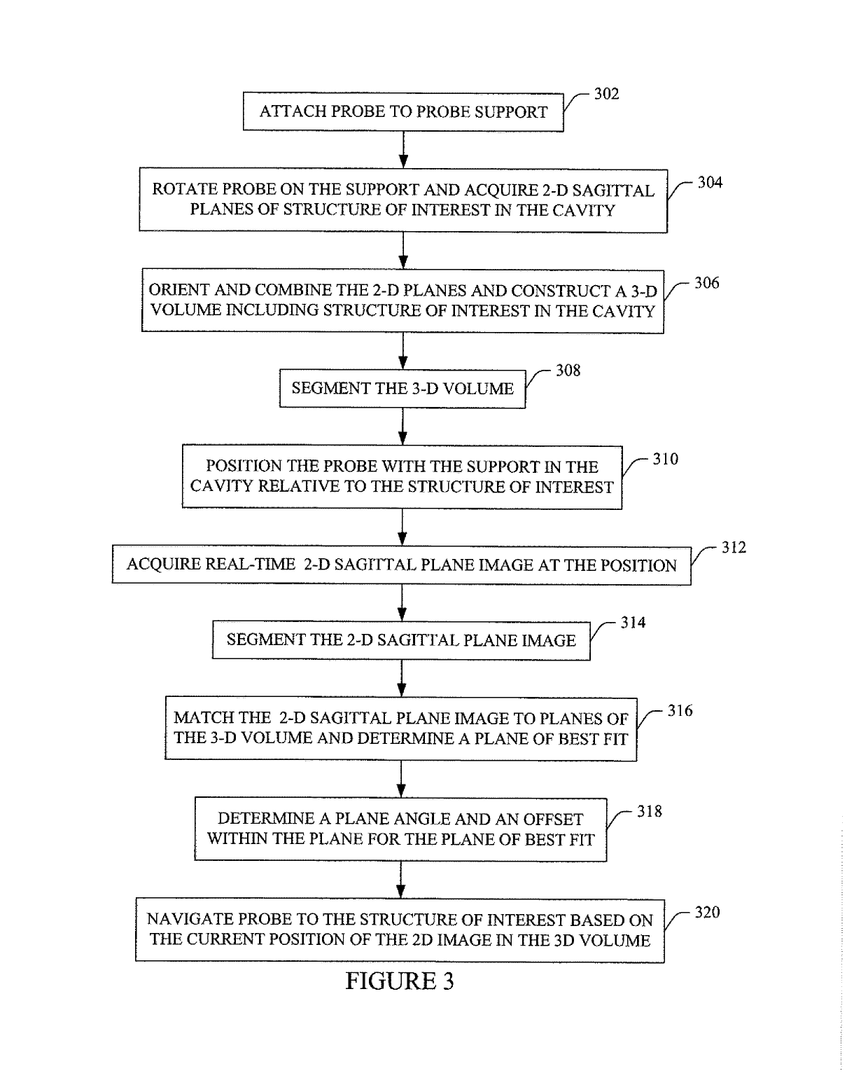

FIG. 3 illustrates an example method using the probe support 116 to support and move the probe 116 to structure of interest for an examination.

It is to be appreciated that the ordering of the above acts is not limiting. As such, other orderings are contemplated herein. In addition, one or more acts may be omitted and/or one or more additional acts may be included.

At 302, the probe 102 with at least a sagittal or end-fire array is attached to the probe support 116.

At 304, 2-D ultrasound images of a structure of interest are acquired with the sagittal or end-fire array while the probe 102 is rotated, either manually or automatically on the support 116 about its longitudinal axis and through an arc in a cavity to scan a full extent of the structure of interest.

At 306, the 2-D ultrasound images are oriented and combined to construct the 3-D navigation image data, which includes the structure of interest.

At 308, structure of interest is segmented in the planes of the 3-D navigation image data. In a variation, the act 308 is omitted.

At 310, the probe 102 is translated and/or rotated manually or automatically on support 116 parallel to and/or around the longitudinal axis to position the probe 102 to acquire a real-time 2-D ultrasound image of the structure of interest.

At 312, the real-time 2-D ultrasound image of the structure of interest is acquired at the position with the sagittal or end-fire array.

At 314, structure of interest also in the 3-D volume is segmented in the real-time 2-D ultrasound image. In a variation, the act 314 is omitted.

At 316, the real-time 2-D ultrasound image is matched with planes of the 3-D navigation image data to determine a plane of best fit and a translational offset within the plane of best fit.

Where the real-time 2-D ultrasound image and the 3-D navigation image data are segmented, the segmented structure can additionally or alternatively be matched. Furthermore, the number of planes of the 3-D volume matched with the real-time 2-D ultrasound image can be reduced, e.g., using knowledge of the angle from the axial plane and/or other information.

At 318, a plane angle and offset within the plane are determined based on the best match.

At 320, the probe 102 is navigated by locating it relative to the structure of interest in the 3-D navigation image data based on the determined plane angle and offset and moving it to the target anatomy based thereon.

For this, in one instance, the real-time 2-D ultrasound image is superimposed over the 3-D navigation image data at the angle and offset and the combination is visually displayed. In a variation, graphical indicia (e.g., an arrow, a schematic of the probe, etc.) is overlaid over the 3-D navigation image data based on the angle and offset.

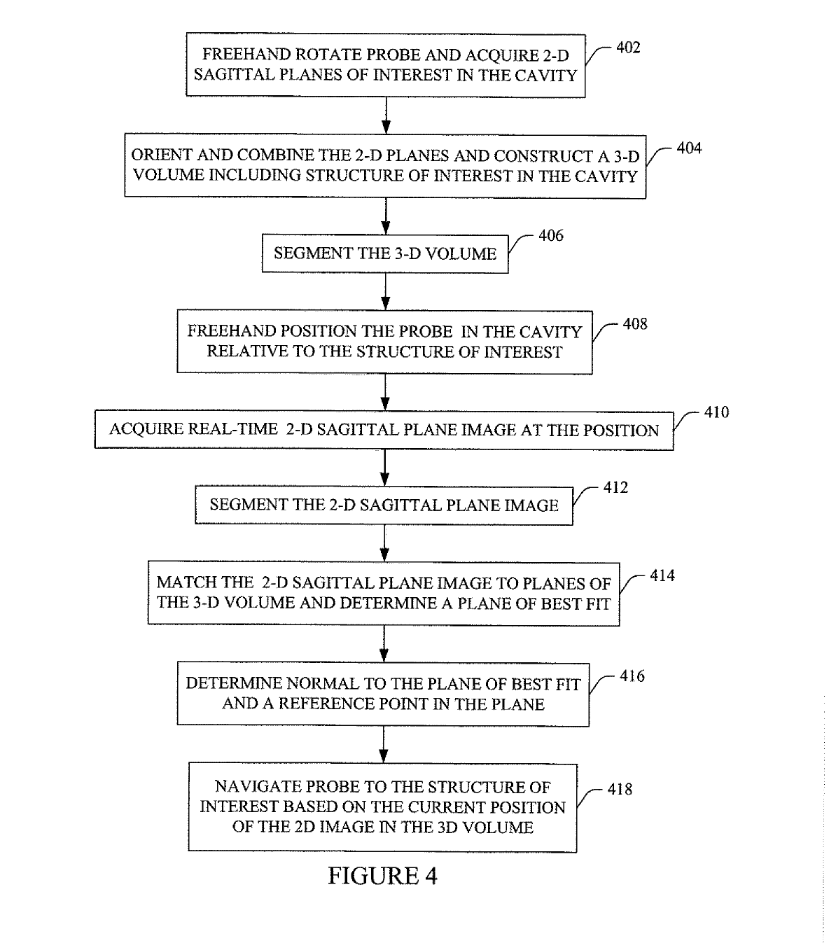

FIG. 4 illustrates an example method a freehand approach to support and move the probe 116 to structure of interest for an examination.

It is to be appreciated that the ordering of the above acts is not limiting. As such, other orderings are contemplated herein. In addition, one or more acts may be omitted and/or one or more additional acts may be included.

At 402, 2-D ultrasound images of a structure of interest are acquired with the sagittal or end-fire array while the probe 102 is freehand rotated about its longitudinal axis through an arc in a cavity.

At 404, the 2-D ultrasound images are oriented and combined to construct the 3-D navigation image data, which includes the structure of interest.

At 406, structure of interest is segmented in the planes of the 3-D navigation image data. In a variation, the act 406 is omitted.

At 408, the probe 102 is freehand translated and/or rotated to position the probe 102 to acquire a real-time 2-D ultrasound image of the structure of interest.

At 410, the real-time 2-D ultrasound image of the structure of interest is acquired at the position with the sagittal or end-fire array.

At 412, structure of interest also in the 3-D volume is segmented in the real-time 2-D ultrasound image. In a variation, the act 412 is omitted.

At 414, the real-time 2-D ultrasound image is matched with planes of the 3-D navigation image data to determine a plane of best fit and a translational offset within the plane of best fit. For anything but small angular deviations from the original rotation axis, this includes interpolating the original planes to a volume, unlike the support-based scan where the planes can remain in their original form and matched to the real-time 2D plane.

Where the real-time 2-D ultrasound image and the 3-D navigation image data are segmented, the segmented structure can additionally or alternatively be matched. Furthermore, the number of planes of the 3-D volume matched with the real-time 2-D ultrasound image can be reduced, e.g., using knowledge of the angle from the axial plane and/or other information such as limiting angular deviations from the axis of the cavity.

At 416, the normal to the plane of best fit and a reference point in the plane corresponding to a known reference point in the 2D sagittal ultrasound image, for example the upper left corner, which positions the 2D sagittal ultrasound image within the 3D volume. are determined.

At 418, the probe 102 is navigated by locating it relative to target anatomy in the 3-D navigation image data based on the position, as determined by the normal vector and point in the best fit 3D plane and moving it to the target anatomy based thereon.

For this, in one instance, the real-time 2-D ultrasound image is superimposed over the 3-D navigation image data at the angle and offset and the combination is visually displayed. In a variation, graphical indicia (e.g., an arrow, a schematic of the probe, etc.) is overlaid over the 3-D navigation image data based on the angle and offset.

At least a portion of the methods discussed herein may be implemented by way of computer readable instructions, encoded or embedded on computer readable storage medium (which excludes transitory medium), which, when executed by a computer processor(s), causes the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A method, comprising:
obtaining a 3-D volume of anatomy including at least a structure of interest;
acquiring, with an array of an ultrasound probe, a real-time 2-D ultrasound sagittal image of the structure of interest in a cavity parallel to a longitudinal axis of the ultrasound probe;
calculating a metric from a 2-D plane extracted from the 3-D volume and the real-time 2-D ultrasound sagittal image, wherein the metric identifies a plane, from sagittal planes of the 3-D volume, and a position within the plane, that best fits the real-time 2-D ultrasound sagittal image; and
identifying a current location of the ultrasound probe with respect to the anatomy based on the identified position.

2. The method of claim 1, further comprising:
navigating the probe to the structure of interest based on the current location of the probe in the 3-D volume.

3. The method of claim 1, further comprising:
visually displaying the 3-D volume with the real-time 2-D ultrasound sagittal image superimposed thereover at the identified plane and the identified position.

4. The method of claim 1, further comprising:
visually displaying the 3-D volume with graphical indicia overlaid at the identified plane and the identified position.

5. The method of claim 1, wherein the 3-D volume includes anatomical structure, and further comprising:
segmenting anatomical structure in the real-time 2-D ultrasound sagittal image corresponding to the included anatomical structure segmented in the 3-D volume; and
matching the common segmented anatomical structure in the real-time 2-D ultrasound sagittal image and the 3-D volume to identify the plane and the position.

6. The method of claim 1, wherein the probe includes an end-fire array, and further comprising: acquiring the real-time 2-D ultrasound sagittal image with the end-fire array.

7. The method of claim 1, wherein the probe includes a biplane probe with a sagittal array and an axial array, and further comprising: acquiring the real-time 2-D ultrasound sagittal image with the sagittal array.

8. The method of claim 7, further comprising:
generating an axial image with data acquired with the axial array; and
determining a similarity between the axial image and a corresponding axial plane in the 3-D volume at the position to validate the identified position.

9. The method of claim 7, further comprising:
interpolating an axial image from the 3-D volume; and
determining a similarity between the axial image and a corresponding axial plane in the 3-D volume at the position to validate the identified position.

10. The method of claim 7, further comprising:
determining a subset of the planes of the 3-D volume to match using an angle derived from the axial image.

11. The method of claim 1, further comprising:
positioning the probe to acquire the real-time 2-D ultrasound sagittal image by translating and rotating the probe to a position of interest with a probe support supporting the probe.

12. The method of claim 1, further comprising:
positioning the probe to acquire the 3-D volume by translating and rotating the probe to a location where an entirety of the structure of interest is visible in the image and rotating the probe through an angular range sufficient to span the volume of the structure of interest with a probe support supporting the probe.

13. The method of claim 1, further comprising:
freehand positioning the probe to acquire the real-time 2-D ultrasound sagittal image.

14. The method of claim 1, further comprising:
freehand positioning the probe to acquire the 3-D ultrasound volume.

15. The method of claim 1, further comprising:
rotating the probe about its longitudinal axis;
transmitting ultrasound signals and receiving echo signals concurrently with the rotating or the translating the first transducer array;
generating spatially sequential 2-D images of the structure of interest with the received echo signals for the plurality of the angles;
identifying the plurality of the angles;
orienting the 2-D images based on the identified plurality of the angles or the linear displacements; and
combining the aligned 2-D images to construct the 3-D volume.

16. The method of claim 1, wherein the matching includes matching the real-time 2-D ultrasound sagittal image with sagittal planes of the 3-D volume based on a similarity metric.

17. The method of claim 1, wherein the matching includes cross-correlating the real-time 2-D ultrasound sagittal image and the sagittal planes of the 3-D volume.

18. An apparatus, comprising:
a sagittal or end-fire transducer array of an ultrasound probe, wherein the sagittal or end-fire transducer array is configured to transmit and receive echoes;
a beamformer configured to process the echoes and generate a real-time 2-D sagittal or end-fire ultrasound image; and
a navigation processor configured to calculate a metric, from a 2-D plane extracted from a 3-D volume and the real-time 2-D ultrasound sagittal or end-fire image, to identify a plane, from sagittal or end-fire planes of the 3-D volume, and a position within the plane, that best fits the real-time 2-D ultrasound sagittal or end-fire image.

19. The apparatus of claim 18, further comprising:
an axial transducer array of the ultrasound probe, wherein the navigation processor further generates an axial image with data acquired with the axial array, and matches the axial image with a corresponding axial plane in the 3-D volume at the position to confirm the identified position.

20. The apparatus of claim 18, wherein the navigation processor further interpolates an axial image from the 3-D volume and matches the axial image with a corresponding axial plane in the 3-D volume at the position to confirm the identified position.

21. The apparatus of claim 19, wherein the navigation processor further determines a subset of planes of the 3-D volume to match using an angle derived from the axial image.

22. The apparatus of claim 18, wherein navigation processor further matches segmented anatomy common in both the real-time 2-D ultrasound image and the 3-D volume to match the real-time 2-D ultrasound image with the planes.

23. The apparatus of claim 18, further comprising:
a probe support configured to support the probe.

24. A non-transitory computer readable medium encoded with computer executable instructions, which, when executed by a computer processor, causes the processor to:
calculate a metric from a 2-D plane extracted from the 3-D volume and a real-time 2-D ultrasound sagittal image, wherein the metric identifies a plane, from sagittal planes of the 3-D volume, and a position within the plane, that best fits the real-time 2-D ultrasound sagittal image; and
identify a current location of the ultrasound probe based on the identified position.
